(19) United States
(12) Patent Application Publication
(10) Pub. No.: US 2023/0173150 A1
(43) Pub. Date: Jun. 8, 2023

(54) Telescopic Plunger for Minimizing Human Breast Milk Fat Loss in a Combination Collection Container and Syringe

(71) Applicant: Avent, Inc., Alpharetta, GA (US)

(72) Inventors: Emily Brayman, Atlanta, GA (US); Caroline M. Alting, Atlanta, GA (US); Oriana M. Bastidas, Atlanta, GA (US); Shaefali M. Padiyar, Atlanta, GA (US); Kiran S. Varadarajan, Atlanta, GA (US)

(21) Appl. No.: 18/075,706

(22) Filed: Dec. 6, 2022

Related U.S. Application Data
(60) Provisional application No. 63/286,142, filed on Dec. 6, 2021.

(51) Int. Cl. A61M 1/06; A61J 1/14; A61M 1/00

(52) U.S. Cl. CPC A61M 1/064; A61J 1/1412; A61M 1/81; A61J 1/1493

(57) ABSTRACT

An apparatus includes a telescopic plunger extending between a proximal end and a distal end. The telescopic plunger includes a plurality of telescoping segments. The plurality of telescoping segments are secured together in an end-to-end configuration and expandable between a collapsed position and an expanded position. The apparatus further includes a collection container secured to the distal end of the telescopic plunger. The collection container defines a barrel having at least one inner wall. The collection container is configured to hold a liquid containing fat molecules therein. As such, a distal-most telescoping segment of the plurality of telescoping segments of the telescopic plunger is configured to sweep the inner wall(s) of the collection container to remove the fat molecules from the inner wall(s) of the collection container.

### RELATED APPLICATIONS

This application claims priority to U.S. Provisional Application No. 63/286,142 filed on Dec. 6, 2021, which is incorporated herein by reference in its entirety.

### FIELD

The present disclosure relates generally to a telescopic plunger for minimizing human breast milk fat loss in a combination collection container and syringe.

### BACKGROUND

The Neonatal Intensive Care Unit (NICU) is a hospital nursery where infants receive specialized care as a result of preterm birth defined as less than 37 weeks of gestation, low birth weight, or health conditions that require continuous care. Out of the 140 million infants that are born each year worldwide, 15 million are born preterm, increasing their susceptibility to health challenges as a consequence of hindered intrauterine development. Ensuring proper nutrition for these patients is of the utmost importance to allow for normal growth and organ development and overcoming preterm health conditions.

In the NICU, growth of the premature infant is critical. The best nutrition to help an infant grow is Human Breast Milk (HBM). Breast milk is uniquely suited to the infant's nutritional needs and is a live substance with unparalleled immunological and anti-inflammatory properties that protect against a host of illnesses and diseases for both mothers and babies. The nutrition profile of human breast milk is composed of several vital macromolecules including lipids, proteins, and carbohydrates. Of these, the largest potential for ensuring maximum nutritional uptake from HBM is lipids. While they make up only 5% of human milk volume, they account for 50% of infants' daily energy intake. Premature babies can absorb 95% of human milk fat; however, on average only 5% of HBM is fat and fat-soluble components, including many vitamins. These fat and fat-soluble components constitute the majority of the calories in HBM, so it is critical to maximize lipid (i.e., fat) delivery.

Studies have shown that due to its molecular properties, fat has a tendency to adhere to the vessels it passes through the complex handling and delivery process. Moreover, many neonate patients are unable to be directly breastfed HBM due to factors such as an underdeveloped digestive system, the presence of ventilators, or an overall lack of physical strength. As a result, providing nutrition via enteral feeding can be necessary.

To optimize neonatal fat delivery, there is a need to minimize fat loss along the enteral feeding process used for premature infants in the NICU. Fat loss in HBM can occur as a consequence of the lengthy process of collecting, measuring, dispensing, and enterally feeding a neonate. Current methods prevent fat loss by products such as an offset syringe, methods to purge the line, various feeding tube materials, and relying on specific feeding methods to deliver HBM. However, in each of these methods there are still many opportunities for fat loss.

Consequently, there is a need for an improved collection container and/or syringe to minimize HBM fat loss during a feeding process, such as enteral feeding. In particular, an improved collection container and/or syringe having improved design for storage and transport would also be useful.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a flow chart of an example process of collecting human breast milk and delivering said breast milk to a neonate according to conventional construction;

FIG. 2 illustrates a perspective view of a telescopic plunger for minimizing human breast milk fat loss in a combination collection container and syringe according to the present disclosure;

FIGS. 3A-3H illustrate perspective views of an embodiment of a plurality of components of a telescopic plunger for minimizing human breast milk fat loss in a combination collection container and syringe according to the present disclosure;

FIG. 4 illustrates a perspective view of a storage cap of a telescopic plunger for minimizing human breast milk fat loss in a combination collection container and syringe according to the present disclosure;

FIG. 5 illustrates a perspective view of an embodiment of a plurality of telescoping segments of a telescopic plunger for minimizing human breast milk fat loss in a combination collection container and syringe according to the present disclosure, particularly illustrating the plurality of telescoping segments in a line;

FIGS. 6A-6C illustrate front views of different embodiments of a plurality of telescoping segments of a telescopic plunger for minimizing human breast milk fat loss in a combination collection container and syringe according to the present disclosure, particularly illustrating different shaped grooves in the plurality of telescoping segments;

FIG. 7 illustrates a perspective view of an apparatus for minimizing human breast milk fat loss in a combination collection container and syringe according to the present disclosure, particularly illustrating a telescopic plunger in a collapsed position and arranged within a storage cap and secured at a distal end of a collection container;

FIG. 8 illustrates a perspective view of an apparatus for minimizing human breast milk fat loss in a combination collection container and syringe according to the present disclosure, particularly illustrating a telescopic plunger in an expanded position and secured at a distal end of a collection container; and

FIG. 9 illustrates a perspective view of an apparatus for minimizing human breast milk fat loss in a combination collection container and syringe according to the present disclosure, particularly illustrating a telescopic plunger in an expanded position and being pushed through a collection container for removing fat molecules on an inner surface of a barrel of the collection container.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

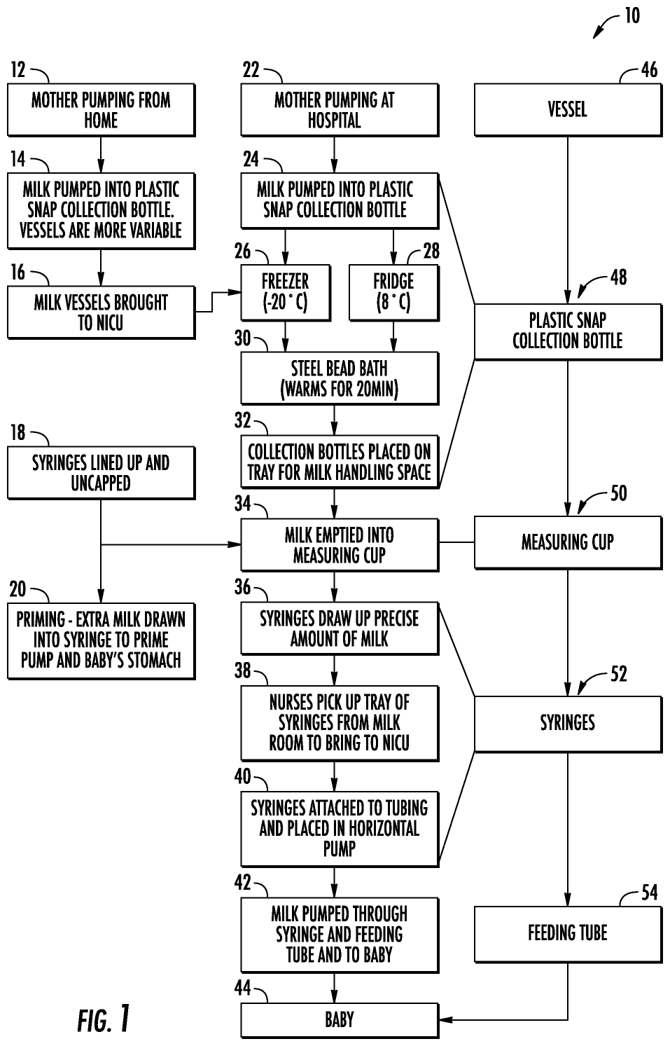

The present disclosure is directed to an apparatus for mitigating fat loss in the enteral feeding system for infants in the NICU. Ensuring proper nutrition for infants in the NICU is of the utmost importance to allow for normal growth and organ development and overcoming preterm health conditions. However, many of these patients are unable to be breastfed HBM due to factors such as an underdeveloped digestive system, the presence of ventilators, or an overall lack of physical strength. As such, if an infant is unable to be breastfed, HBM must be expressed and provided to the infant using a feeding apparatus, such as a syringe or bottle. An embodiment of a process 10 of collecting HBM is illustrated in FIG. 1. In particular, as shown at 12, the process 10 includes a mother pumping HBM from home. As shown at 14, the HBM may be pumped into a plastic container, such as a plastic snap collection bottle, however, such collection vessels are variable. As shown at 16, the process 10 includes transporting the milk vessels to the NICU.

As shown at 22 and 24, the mother may also pump the HBM into a vessel 46, such as a plastic snap collection bottle 48, at the hospital. Thus, as shown at 26 and 28, the process 10 includes placing the pumped HBM into a freezer or a refrigerator. When it is time to feed the infant, as shown at 30, the vessel 46 can be placed into a steel bead bath for warming. For example, the vessel 46 may be heated for about 20 minutes. After warming, as shown at 32, the process 10 includes placing the collection bottles on a tray for milk handling space. As shown at 34, the process 10 then includes emptying the milk into a measuring cup 50. As shown at 36, the process 10 includes drawing up a precise amount of the milk into a syringe 52. As shown at 38, the process includes having a nurse pick up the tray of syringes from a milk room for delivering to the NICU. As shown at 40, the process includes attaching the syringes to tubing and/or placing in a horizontal pump. As shown at 42, the milk can then be pump through the syringe through a feeding tube into the infant. As shown at 44, the baby is able to eat the HBM.

Accordingly, due to the lengthy process of expressing HBM and delivering the expressed HBM to the infant, too much fat loss in the HBM can occur. In order to optimize neonatal fat delivery, the present disclosure is directed to an apparatus for minimizing fat loss along the enteral feeding process used for premature infants in the NICU. In particular, the present invention is directed to an apparatus having a unique telescopic collapsible plunger used in combination with a collection container, e.g., collection bottle. As such, the telescopic plunger is configured to replicate the sweeping movement that is typically incorporated into an enteral feeding syringe to sweep the walls of the collection container of fat so the fat can be easily transferred to the preparation beaker or syringe for subsequent preparation steps. The collection bottle can be then used as just that and/or can be used as a syringe, if desired. The telescopic plunger may be useful to collapse for storage. Further, in an embodiment, the telescopic plunger may utilize the benefits of a syringe to push all of the fat from the collection container with an offset opening. Moreover, the telescopic plunger can be collapsed to a storage arrangement so that the telescopic plunger may sit on a shelf and be stored and transported easily.

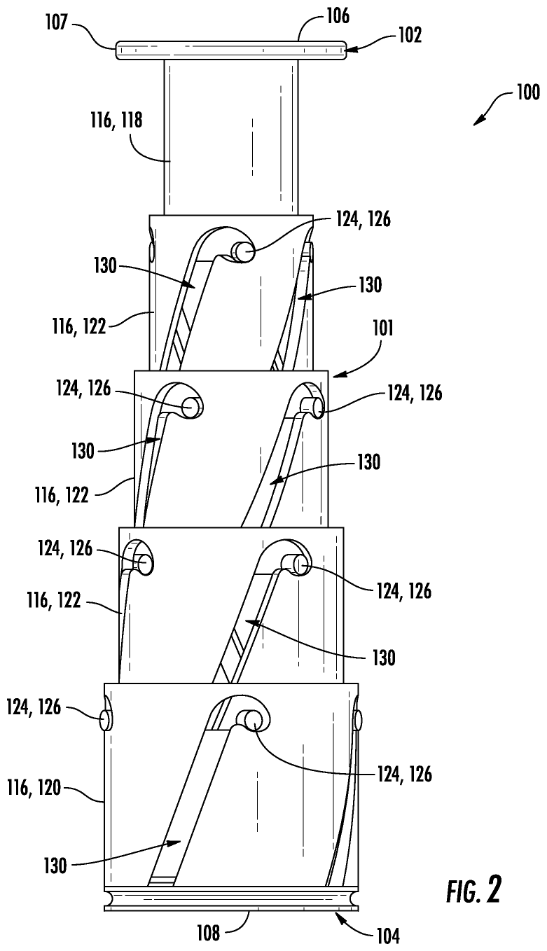

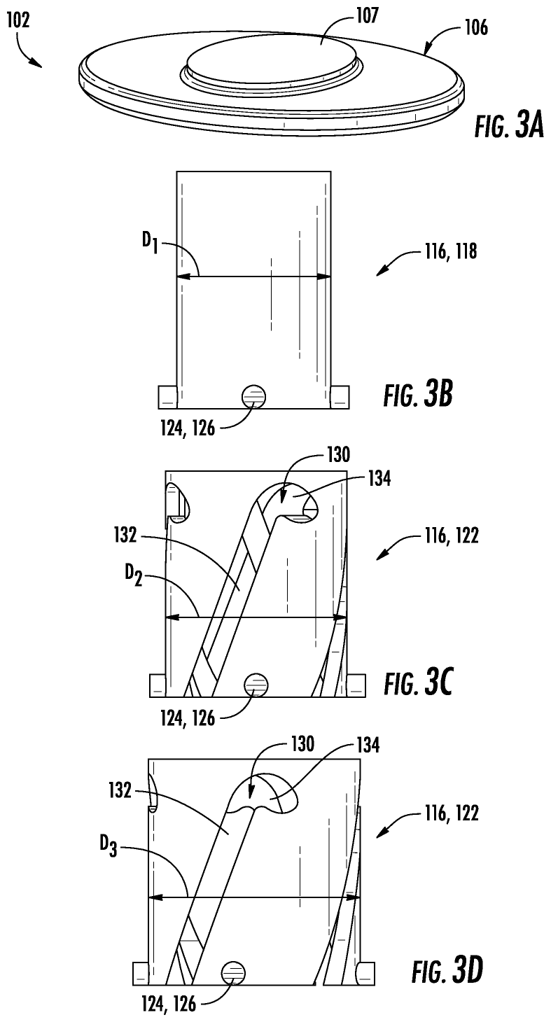

Referring now to FIGS. 2-9, various views of an apparatus 100 for maximizing fat transfer from HBM to infants according to the present disclosure are illustrated. In particular, as shown, the apparatus 100 includes a telescopic plunger 101 according to the present disclosure are illustrated. In particular, as shown in FIG. 2, the telescopic plunger 101 can have a proximal end 102 and a distal end 104. As shown particularly in FIG. 3A, the proximal end 102 may include a plunger base 106. Further, as shown, the plunger base 106 may include a handle 107 configured to be grasped by a user.

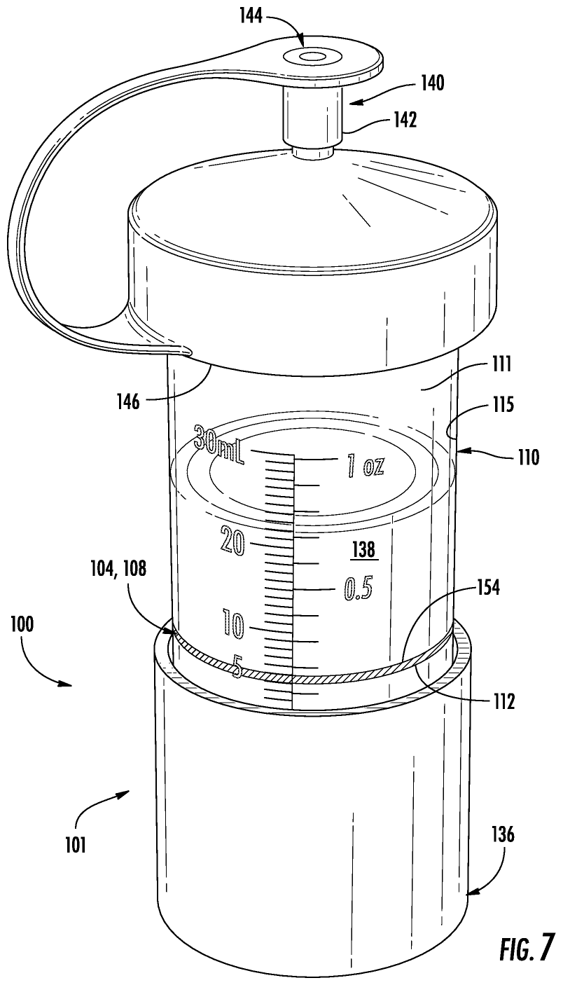

Furthermore, as shown in FIGS. 2, 3G, and 8, the distal end 104 can include a plunger head 108 configured to be inserted within a barrel 111 of a collection container 110 (see e.g., FIGS. 7-9). Thus, in embodiment, the telescopic plunger 101 may be housed within a cap (e.g., a storage cap 136) of the collection container 110. As shown particularly in FIGS. 3G and 3H, the plunger head 108 can include a seal 112 configured to provide a fluid-tight seal when the plunger head 108 is inserted within the barrel 111 of the collection container 110. For instance, as shown in FIG. 3H, the seal 112 can be in the form of an O-ring 117 or any other suitable sealing feature. Moreover, in an embodiment, as shown in FIG. 3G, for example, the plunger head 108 can have a generally cylindrical-shaped body 114 having a concave indentation 116 extending around a circumference of the cylindrical-shaped body 114. In particular embodiments, for example, the concave indentation 116 can be configured to receive the O-ring 117 (see e.g., FIG. 3H). The seal 112 can be formed from rubber or any other suitable material. Moreover, the O-ring 117 is configured to tightly engage an inner surface 115 of the barrel 111 of the collection container 110 (see e.g., FIGS. 7-9) to sweep the inner surface 115 of the barrel 111 in order to prevent fat (lipid) molecules from becoming stuck thereto. In this regard, the seal 112, e.g., the O-ring 117, can directly increase the amount of fat (lipids) delivered to a patient via the syringe by sweeping the inner surface 115 of the barrel 111 to push fat (lipid) molecules toward a syringe tip 142 (as further explained in detail herein below).

Referring now to FIGS. 2, 3B-3F, and 5, the telescopic plunger 101 includes a plurality of telescoping segments 116 extending between the proximal end 102 and the distal end 104. For instance, in an embodiment, the telescopic plunger 101 may include at least two telescoping segments 116. In further embodiments, the telescopic plunger 101 may include more than two telescoping segments 116, e.g., from two telescoping segments 116 to ten or more telescoping segments 116. In particular embodiments, as shown in FIGS. 2, 3B-3F, and 5, for example, the telescopic plunger 101 may include five telescoping segments 116. Furthermore, as shown in FIG. 2, the plunger base 106 can be coupled to a proximal-most telescoping segment 118. Moreover, as shown in FIG. 2, the plunger head 108 can be coupled to a distal-most telescoping segment 120.

Referring particularly to FIGS. 2, 3B-3F, and 5, each telescoping segment 116 can have a different diameter than each other telescoping segment 116. For example, in an embodiment, as shown in FIGS. 3B, the proximal-most telescoping segment 118 can have a first diameter (labeled as Di) that is smaller than a diameter of any other telescoping segment 116 (labeled as D2, D3, D4, D5). Furthermore, in an embodiment, as shown in FIG. 3F, the distal-most telescoping segment 120 can have a second diameter (labeled as D5) that is larger than a diameter of any other telescoping segment 116 (labeled as D1, D2, D3, D4). The respective diameter of each intermediate telescoping segment 122 disposed between the proximal-most telescoping segment 118 and the distal-most telescoping segment 120 (labeled as D2, D3, D4) can be generally increasing such that each of the telescoping segments 116 can be nested inside each other when the telescopic plunger 101 is in a collapsed position (see e.g., FIG. 7). In further embodiments, the present disclosure further contemplates any suitable arrangement of telescoping segments 116 having any desired diameter. For instance, the diameter of the telescoping segments 116 can decrease from the proximal end 102 to the distal end 104.

Referring now to FIGS. 2, 3B-3E, 5, and 6A-6C, some or all of the telescoping segments 116 can include engagement features 124 configured to couple to an adjacent telescoping segment 116. In certain embodiments, the engagement features 124 can be equally disposed about the circumference of the telescoping segments 116. In alternative embodiments, the engagement features 124 can be placed in any desired relative position about the circumference of the telescoping segments 116. In further embodiments, the telescopic plunger 101 may include four engagement features 124 respectively disposed about the circumference about each telescoping segment 116. In additional embodiments, the telescopic plunger 101 may include more than four or less than four engagement features 124 disposed about the circumference about each telescoping segment 116.

In further embodiments, as shown in FIGS. 2, 3B-3E, 5, and 6A-6C, some or all of the telescoping segments 116 can include one or more pins 126 extending from an outer surface 128 of the telescoping segment 116. Additionally, or alternatively, the pin(s) 126 can extend from an inner surface of any of the telescoping segments 116.

Accordingly, as shown in FIGS. 2, 3C-3F, 5, and 6A-6C, the pin(s) 126 can be received in a corresponding groove 130 of an adjacent telescoping segment 116. Furthermore, as shown particularly in FIGS. 3C-3F, each groove 130 can include an elongated portion 132 extending in a generally axial direction of the telescopic plunger 101 and a locking portion 134 extending from the elongated portion 132 in a generally radial direction of the telescopic plunger 101. Thus, the pin(s) 126 of a given telescoping segment 116 can extend within the groove(s) 130 of an adjacent telescoping segment 116 such that each pin 126 can slide along a respective elongated portion 132 and be locked in place when the pin 126 is disposed in the corresponding locking portion 134. Moreover, in an embodiment, when the telescoping plunger 101 is in an extended position (see e.g., FIG. 2), each of the pins 126 may be disposed in a respective corresponding locking portion 134. To collapse the telescoping plunger 101 to a collapsed or retracted position (see e.g., FIG. 7), each of the pins 126 can be slid out from their respective locking portions 134 and slid along the elongated portions 132 of each respective groove 130.

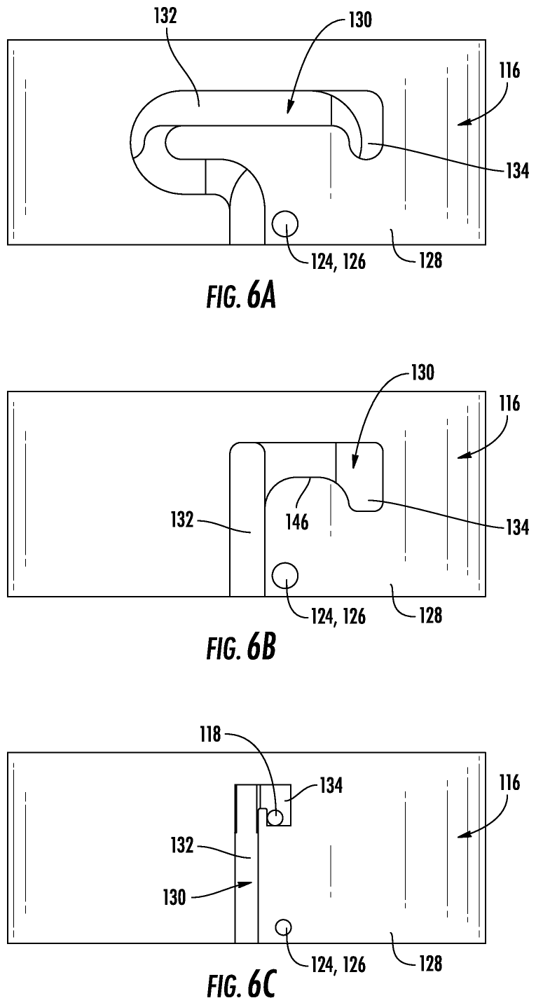

Moreover, and referring to FIGS. 6A-6C, the grooves 130 may have any suitable shape. In particular, as shown in FIGS. 6A and 6B, the grooves 130 may have a generally J-shaped configuration. In addition, as shown, the grooves 130 may be oriented in any direction (e.g., longitudinally or laterally) on the outer surface of a given telescoping segment 116. In another embodiment, as shown in FIG. 6B and 6C, the grooves 130 may have a generally L-shaped configuration. Furthermore, as shown, the L-shaped groove 130 may have a filleted portion 146. In further embodiments, the groove(s) 130 may have any other suitable shape, such as an S-shape.

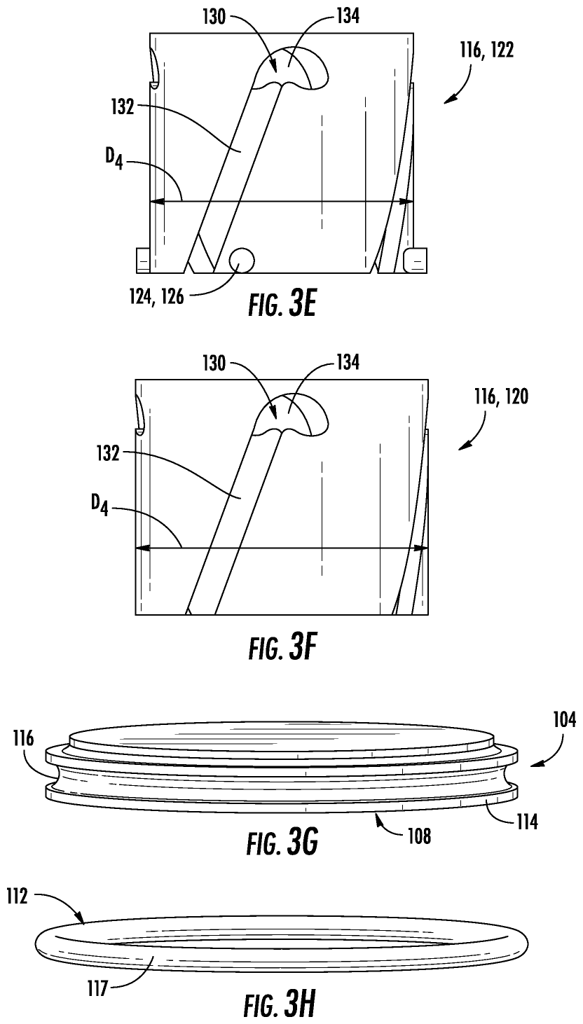

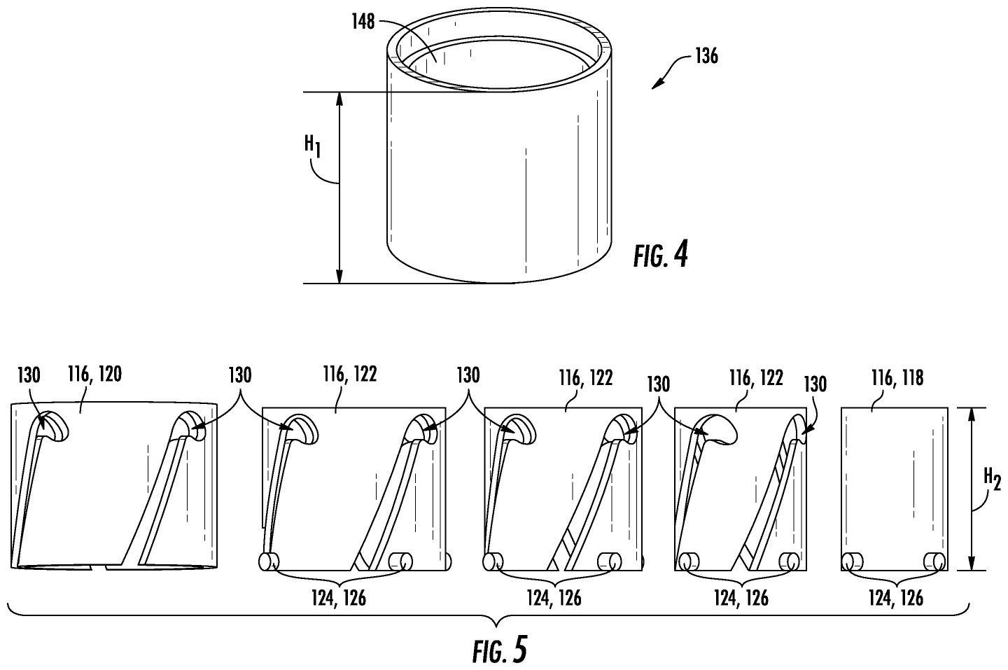

Referring now to FIGS. 4 and 7, the telescopic plunger 101 can further include a storage cap 136. In an embodiment, for example, as shown in FIG. 7, the storage cap 136 can be configured to receive the telescopic plunger 101 when the telescopic plunger 101 is in a collapsed position. For example, as shown in FIGS. 4 and 5, the storage cap 136 may generally define a first height Hi, whereas each of the telescoping segments 116 may generally define a second height H2. Thus, the first and second heights H1, H2 may be generally equal such that the telescoping segments 116 are nested within each other when the telescopic plunger 101 is in the collapsed position. As such, the nested telescoping segments 116 can be easily received within the storage cap 136. More specifically, in an embodiment, the storage cap 136 can be configured to receive the proximal end 102 of the telescopic plunger 101 within a cavity 148 thereof (FIG. 4) (e.g., by placing the proximal end 102 of the telescopic plunger 101 into the cavity 148 first) with the plunger head 108 of the telescopic plunger 101 extending out of the storage cap 136 and into the collection container 110. Thus, in an embodiment, the plunger head 108 may or may not be disposed within the storage cap 136.

Referring now to FIGS. 7-9, the telescopic plunger 101 of the present disclosure can be used in connection with a syringe 140 for delivering HBM 138 to a neonate via enteral feeding. In particular, as shown in FIG. 7, the syringe 140 can be formed in combination with the collection container 110. Thus, as shown in FIG.

9, in an embodiment, the syringe 140 may include a syringe tip 142 covered by a removable syringe cap 144. Accordingly, in an embodiment, when using the apparatus 100, HBM 138 can be delivered, e.g., via a breast pump, into the collection container 110. Further, as shown in FIG. 7, the telescopic plunger 101 can be housed within the storage cap 136 in a collapsed position and secured at its distal end 104 to a distal end 154 of the collection container 110 containing the HBM 138. Furthermore, in an embodiment, the telescopic plunger 101, in its collapsed position, can form a cap at the distal end 154 of the collection container 110.

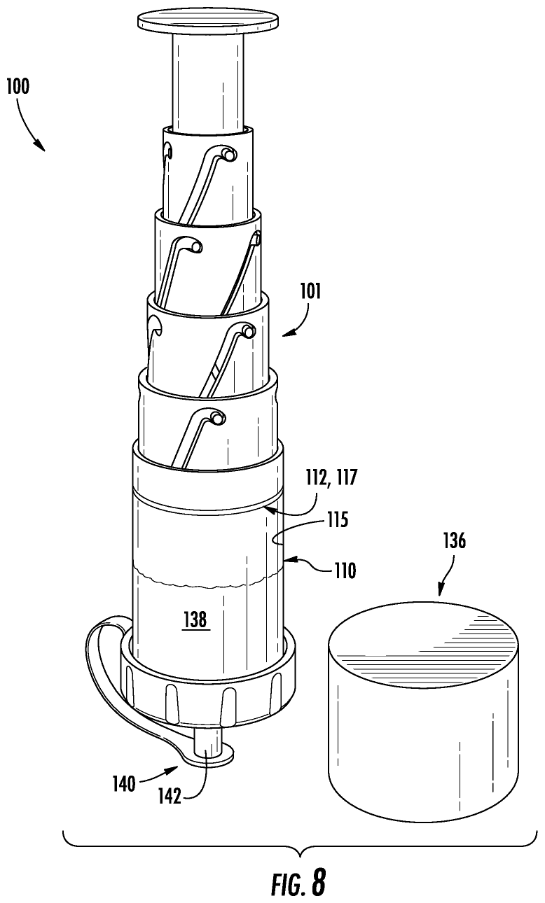

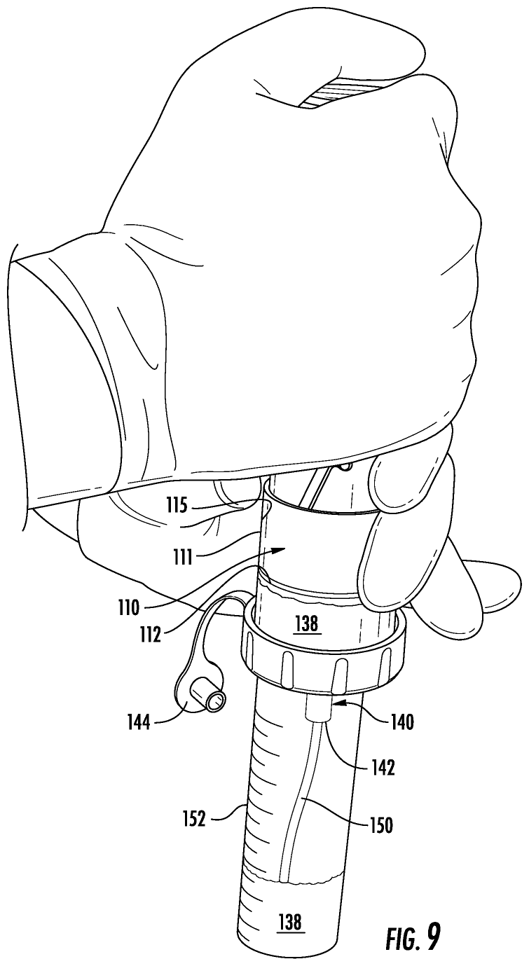

Referring particularly to FIG. 8, the storage cap 136 can be removed and the telescopic plunger 101 may be inverted and expanded to its expanded position (e.g., by engaging the handle 107 of the plunger base 106 and pulling on the telescopic plunger 101 to expand the telescoping segment 116). Moreover, in an embodiment, the telescopic plunger 101 can be locked or otherwise secured in its expanded position using the pins 126 and grooves 130 described herein. Once locked, the telescopic plunger 101 can be engaged by a user and pushed through the collection container 110. Thus, as shown in FIG. 9, the syringe cap 144 can be removed from the syringe 140 to expose the syringe tip 142. Then, in an embodiment, the telescopic plunger 101 can be engaged by the user as a syringe plunger to deliver the HBM 138 to a feeding tube 150 through the syringe tip 142. In other words, the collection container 110 also works as a syringe to deliver the HBM 138 directly to the neonate via the feeding tube 150.

In another embodiment, and referencing FIG. 7, HBM 138 and/or fortifiers may be added to the collection container 110 prior to use, thereby causing the distal end 104 of the telescopic plunger 101 to move downward. Such fortifiers, for example, are beneficial in medical settings that batch milk for 12 or 24 hours of feeding. In such embodiments, once the HBM 138 and potential fortifiers are added to the collection container 110, they are mixed together and can be drawn back up using a syringe (not shown). Thus, in an embodiment, the moveable distal end 104/seal 112 scraps the sides of the walls of the collection container 110 to remove the fat therefrom.

By using a combination collection container and syringe, along with the telescopic plunger 101, to deliver the HBM 138 to a neonate through the enteral feeding tube 150, approximately 96% of the fat (lipids) in the HBM collected in the collection container are successfully transferred to the neonate. In other words, as shown in FIG. 9 and mentioned, the seal 112 is configured to tightly engage an inner surface 115 of the barrel 111 of the collection container 110 to sweep the inner surface 115 of the barrel 111 in order to prevent fat (lipid) molecules from becoming stuck thereto. In this regard, the seal 112 can directly increase the amount of fat molecules delivered to the neonate. In contrast, when using a conventional system which requires transferring HBM from a collection container to a syringe, and possibly including transferring HBM to a measuring cup prior to transferring to a syringe, only about 70% of fat (lipids) in the HBM collected in the collection container are successfully transferred to the neonate.

In other words, by at least decreasing the number of vessels needed to collect and deliver HBM to a neonate, the apparatus 100 of the present disclosure is able to transfer HBM having a significantly higher fat (lipid) content (e.g., more than about 25% more fat (lipids) delivered to the neonate as compared with conventional systems). Moreover, reducing the number of vessels required for the enteral feeding process using the apparatus 100 of the present disclosure directly increases the ease of use for nurses, milk technicians, and other care providers in the enteral feeding process.

While the telescopic plunger 101 has been described above specifically with regard to collection and enteral feeding of HBM, the present disclosure also contemplates that the telescopic plunger 101 and/or the storage cap 136 can be used in a variety of clinical and non-clinical applications, such as enteral feeding of non-HBM substances, medication or pharmaceutical delivery, vascular applications, pain-management applications, or any other desirable use for which the present invention may be deemed appropriate.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. An apparatus, comprising:
a telescopic plunger extending between a proximal end and a distal end, the telescopic plunger comprising a plurality of telescoping segments, the plurality of telescoping segments secured together in an end-to-end configuration and expandable between a collapsed position and an expanded position; and
a collection container secured to the distal end of the telescopic plunger, the collection container defining a barrel comprising at least one inner wall, the collection container configured to hold a liquid containing fat molecules therein,
wherein a distal-most telescoping segment of the plurality of telescoping segments of the telescopic plunger is configured to sweep the at least one inner wall of the collection container to remove the fat molecules from the at least one inner wall of the collection container.

2. The apparatus of claim 1, wherein the proximal end of the telescopic plunger comprises a plunger base and the distal end comprises a plunger head configured to be inserted within the barrel of the collection container.

3. The apparatus of claim 2, wherein the plunger base comprises a handle configured to be grasped by a user.

4. The apparatus of claim 2, wherein the plunger head comprises a seal configured to provide a fluid-tight seal when the plunger head is inserted within the barrel of the collection container, the seal configured to sweep the at least one inner wall of the barrel of the collection container to remove the fat molecules therefrom.

5. The apparatus of claim 3, wherein the seal is an O-ring.

6. The apparatus of claim 4, wherein the plunger head defines a generally cylindrical-shaped body comprising a concave indentation extending around a circumference of the cylindrical-shaped body, the concave indentation configured to receive the O-ring.

7. The apparatus of claim 1, wherein each of the plurality of telescoping segments comprises a different diameter such that the plurality of telescoping segments are nested together in the collapsed position.

8. The apparatus of claim 1, wherein the plurality of telescoping segments each comprise at least one groove and at least one pin, wherein the at least one pin of one of the plurality of telescoping segments is configured to be received in the at least one groove of an adjacent telescoping segment of the plurality of telescoping segments for locking the telescoping plunger in the expanded position.

9. The apparatus of claim 8, wherein the at least one groove of each the plurality of telescoping segments comprises an elongated portion and a locking portion extending from the elongated portion, wherein the at least one pin of one of the plurality of telescoping segments extends in the at least one groove of the adjacent telescoping segment in the expanded position such that the at least one pin slides along the elongated portion and is locked in place when the at least one pin is disposed in the locking portion.

10. The apparatus of claim 8, wherein the at least one groove of each of the plurality of telescoping segments comprises at least one of an L-shape, a J-shape, or an S-shape.

11. The apparatus of claim 1, wherein the collection container defines a proximal end and a distal end, the distal end of the collection container comprising a syringe having a syringe tip.

12. The apparatus of claim 11, further comprising a feeding tube in fluid communication with the syringe tip.

13. The apparatus of claim 11, further comprising a storage cap secured to the distal end of the collection container and acting as a lid to the collection container, wherein the telescopic plunger is at least partially housed within the storage cap.

14. A telescopic plunger, comprising:
a body comprising a plurality of telescoping segments secured together in an end-to-end configuration and expandable between a collapsed position and an expanded position, the body of the telescopic plunger extending between a proximal end and a distal end,
wherein a distal-most telescoping segment of the plurality of telescoping segments of the telescopic plunger is configured to sweep an inner wall of a barrel of a collection container secured at the distal end of the telescopic plunger to remove fat molecules from the at least one inner wall of the collection container.

15. The telescopic plunger of claim 14, wherein the proximal end of the telescopic plunger comprises a plunger base and the distal end comprises a plunger head configured to be inserted within the barrel of the collection container.

16. The telescopic plunger of claim 15, wherein the plunger base comprises a handle configured to be grasped by a user.

17. The telescopic plunger of claim 15, wherein the plunger head comprises a seal configured to provide a fluid-tight seal when the plunger head is inserted within the barrel of the collection container, the seal configured to sweep the at least one wall of the barrel of the collection container to remove the fat molecules therefrom.

18. The telescopic plunger of claim 17, wherein the plunger head defines a generally cylindrical-shaped body comprising a concave indentation extending around a circumference of the cylindrical-shaped body, the concave indentation configured to receive the seal.

19. The telescopic plunger of claim 15, wherein each of the plurality of telescoping segments comprises a different diameter such that the plurality of telescoping segments are nested together in the collapsed position.

20. The telescopic plunger of claim 15, wherein the plurality of telescoping segments each comprise at least one groove and at least one pin, wherein the at least one pin of one of the plurality of telescoping segments is configured to be received in the at least one groove of an adjacent telescoping segment of the plurality of telescoping segments for locking the telescoping plunger in the expanded position.
